(19) United States
(12) Patent Application Publication
(10) Pub. No.: US 2018/0056074 A1
(43) Pub. Date: Mar. 1, 2018

(54) SYSTEMS AND METHODS FOR REVERSIBLE NERVE BLOCK TO RELIEVE DISEASE SYMPTOMS

(71) Applicant: Boston Scientific Scimed, Inc., Maple Grove, MN (US)

(72) Inventors: Bryan A. Clark, Forest Lake, MN (US); Jai Shetake, Santa Clarita, CA (US); Aiden Flanagan, Galway (IE)

(21) Appl. No.: 15/686,867

(22) Filed: Aug. 25, 2017

Related U.S. Application Data
(60) Provisional application No. 62/379,668, filed on Aug. 25, 2016.
(60) Provisional application No. 62/416,255, filed on Nov. 2, 2016.

(51) Int. Cl. A61N 1/36; A61B 5/00; A61B 5/04; A61N 1/05; A61N 1/378; A61N 1/372

(52) U.S. Cl. CPC A61N 1/36185; A61B 5/4836; A61B 5/04001; A61N 1/0556; A61N 1/0551; A61B 5/08; A61N 1/36125; A61N 1/36139; A61N 1/3614; A61N 1/3787; A61N 1/37229; A61N 1/3601

(57) ABSTRACT

The present disclosure relates to the field of neuromodulation. Specifically, the present disclosure relates to systems and methods for reversibly blocking an electrical signal from travelling along a target nerve. In particular, the present disclosure relates to systems and methods for relieving a pulmonary symptom by reversibly blocking an electrical signal from travelling along the vagus nerve or internal branch of the superior laryngeal nerve

### CROSS REFERENCE TO RELATED APPLICATIONS

The present application claims the benefit of priority under 35 U.S.C. §119 to U.S. Provisional Patent Application Ser. No. 62/379,668, filed on Aug. 25, 2016, and U.S. Provisional Patent Application Ser. No. 62/416,255, filed on Nov. 2, 2016, both of which are incorporated by reference in their entireties for all purposes

### FIELD

The present disclosure relates to the field of neuromodulation. Specifically, the present disclosure relates to systems and methods for reversibly blocking an electrical signal from travelling along a target nerve. In particular, the present disclosure relates to systems and methods for relieving a pulmonary symptom by reversibly blocking an electrical signal from travelling along the vagus nerve or internal branch of the superior laryngeal nerve.

### BACKGROUND

Chronic obstructive pulmonary disease (COPD) includes conditions such as, e.g., chronic bronchitis and emphysema. COPD currently affects over 15 million people in the United States alone and is currently the third leading cause of death in the country. The primary cause of COPD is the inhalation of cigarette smoke, responsible for over 90% of COPD cases. The economic and social burden of the disease is substantial and is increasing.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result.

Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Acute exacerbations of COPD (AECOPD) often require emergency care and inpatient hospital care. An AECOPD event is defined by a sudden worsening of symptoms (e.g., increase in or onset of cough, wheeze, and sputum changes) that typically last for several days, but can persist for weeks. An AECOPD event is typically triggered by a bacterial infection, viral infection, or pollutants, which manifest quickly into airway inflammation, mucus hypersecretion, and bronchoconstriction, causing significant airway restriction.

Despite relatively efficacious drugs (long-acting muscarinic antagonists, long-acting beta agonists, corticosteroids, and antibiotics) that treat COPD symptoms, a particular segment of patients known as “frequent exacerbators” often visit the emergency room and hospital with exacerbations and also have a more rapid decline in lung function, poorer quality of life, and a greater mortality risk.

Reversible obstructive pulmonary disease includes asthma and reversible aspects of COPD. Asthma is a disease in which bronchoconstriction, excessive mucus production, and inflammation and swelling of airways occur, causing widespread but variable airflow obstruction thereby making it difficult for the asthma sufferer to breathe. Asthma is further characterized by acute episodes of airway narrowing via contraction of hyper-responsive airway smooth muscle.

The reversible aspects of COPD include excessive mucus production and partial airway occlusion, airway narrowing secondary to smooth muscle contraction, and bronchial wall edema and inflation of the airways. Usually, there is a general increase in bulk (hypertrophy) of the large bronchi and chronic inflammatory changes in the small airways. Excessive amounts of mucus are found in the airways, and semisolid plugs of mucus may occlude some small bronchi. Also, the small airways are narrowed and show inflammatory changes.

In asthma, chronic inflammatory processes in the airway play a central role in increasing the resistance to airflow within the lungs. Many cells and cellular elements are involved in the inflammatory process including, but not limited to, mast cells, eosinophils, T lymphocytes, neutrophils, epithelial cells, and even airway smooth muscle itself. The reactions of these cells result in an associated increase in sensitivity and hyperresponsiveness of the airway smooth muscle cells lining the airways to particular stimuli.

The chronic nature of asthma can also lead to remodeling of the airway wall (i.e., structural changes such as airway wall thickening or chronic edema) that can further affect the function of the airway wall and influence airway hyper-responsiveness. Epithelial denudation exposes the underlying tissue to substances that would not normally otherwise contact the underlying tissue, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance, pharmacology and bronchial thermoplasty.

The autonomic nervous system (ANS) provides constant control over airway smooth muscle, secretory cells, and vasculature. The ANS is divided into two subsystems, the parasympathetic nervous system and the sympathetic nervous system. These two systems operate independently for some functions, and cooperatively for other functions. The parasympathetic system is responsible for the unconscious regulation of internal organs and glands. In particular, the parasympathetic system is responsible for sexual arousal, salivation, lacrimation, urination, and digestion, among other functions. The sympathetic nervous system is responsible for stimulating activities associated with the fight-or-flight response. Although both sympathetic and parasympathetic branches of the ANS innervate lung airways, it is the parasympathetic branch that dominates with respect to control of airway smooth muscle, bronchial blood flow, and mucus secretions.

FIG. 1 illustrates the cholinergic control of airway smooth muscle and submucosal glands. An airway 100 may include an inner surface 102 that includes epithelial tissue 104. Nerve fibers 106 may be C-fibers having a plurality of receptors 108 disposed within epithelial tissue 104. Nerve fibers 106 may be afferent (sensory) nerves that carry nerve impulses from receptors 108 toward central nervous system (CNS) 109. Receptors 108 may respond to a wide variety of chemical stimuli and other irritants, such as, e.g., cigarette smoke, histamine, bradykinin, capsaicin, allergens, and pollens. C-fibers can also be triggered by autocoids that are released upon damage to tissues of the lung. The stimulation of receptors 108 by the various stimuli elicits reflex cholinergic bronchoconstriction.

Parasympathetic innervation of the airways is carried by vagus nerve 110 (e.g., the right and left vagus nerves). Upon receiving an electrical signal from nerve fiber 106, CNS 109 may send an electrical signal to initiate bronchoconstriction and/or mucus secretion. Cholinergic nerve fibers (e.g., nerve fibers that use acetylcholine (ACh) as their neurotransmitter) arise in the nucleus ambiguous in the brain stem and travel down a vagus nerve 110 (right and left vagus nerves) and synapse in parasympathetic ganglia 112 which are located within the airway wall. These parasympathetic ganglia are most numerous in the trachea and mainstem bronchi, especially near the hilus and points of bifurcations, with fewer ganglia that are smaller in size dispersed in distal airways. From these ganglia, short post-ganglionic fibers 114 travel to airway smooth muscle 116 and submucosal glands 118. ACh, the parasympathetic neurotransmitter, is released from post-ganglionic fibers and acts upon M1- and M3-receptors on smooth muscles 116 and submucosal glands 118 to cause bronchoconstriction (via constriction of smooth muscles 116), and the secretion of mucus 122 within airway 100 by submucosal glands 118, respectively. ACh may additionally regulate airway inflammation and airway remodeling, and may contribute significantly to the pathophysiology of obstructive airway diseases. Thus, fibers 114 may be efferent fibers (motor or effector neurons) that are configured to carry nerve impulses away from CNS 109.

FIG. 2 illustrates additional afferent nerve fibers located in airway 100 and in airway smooth muscle 116. Airway 100 may include one or more nerve fibers 106 and receptors 108 as described with reference to FIG. 1. Additionally, one or more nerve fibers 206 having one or more receptors 208 may be disposed within epithelial tissue 104. Nerve fibers 206 may be myelinated Rapidly Adapting Receptors (RAR) that respond to mechanical stimuli and are responsible in part for bronchoconstriction. Receptors 208 may respond to mechanical stimuli such as, e.g., water, airborne particulates, mucus, and the stretching of the lung during breathing or coughing. RARs may cause bronchoconstriction and are triggered by merchant-stimulation (e.g., mechanical pressure or distortion) and/or chemo-stimulation. Additionally, RARs may be triggered secondary to bronchoconstriction, leading to an amplification of the constriction response.

Airway smooth muscle 116 may be coupled to one or more receptors 210. Receptors 210 may be, e.g., Slowly Adapting Receptors (SARs) that are coupled to one or more nerve fibers 211.

Bronchial hyperresponsivity (BHR) may be present in a considerable number of COPD patients. Various reports have suggested BHR to be present in between about 60% and 94% of COPD patients. This “hyperresponsivity” could be due to a “hyperreflexivity.” However, there are several logical mechanisms by which parasympathetic drive may be over-activated in inflammatory disease. First, inflammation is commonly associated with overt activation and increases in excitability of vagal C-fibers in the airways that could increase reflex parasympathetic tone. Secondly, airway inflammation and inflammatory mediators have been found to increase synaptic efficacy and decrease action potential accommodation in bronchial parasympathetic ganglia, effects that would likely reduce their filtering function and lead to prolonged excitation. Thirdly, airway inflammation has also been found to inhibit muscarinic M2 receptor-mediated auto-inhibition of ACh release from postganglionic nerve terminals. This would lead to a larger end-organ response (e.g., smooth muscle contraction) per a given amount of action potential discharge. Fourthly, airway inflammation has been associated with phenotypic changes in the parasympathetic nervous system that could affect the balance of cholinergic contractile versus non-adrenergic non-cholinergic (NANC) relaxant innervation of smooth muscle.

Because airway resistance varies inversely with the fourth power of the airway radius, BHR is believed to be a function of both bronchoconstriction and inflammation. Inflammation in the airway walls reduces the inner diameter (or radius) of the airway lumen, thus amplifying the effect of even baseline cholinergic tone, because for a given change in muscle contraction, the airway lumen will close to a greater extent. BHR is likely caused by hypersensitivity of receptor nerve fibers, such as, e.g., C-fibers, RAR fibers, SAR fibers, and the like, lower thresholds for reflex action initiation, and reduced self-limitation of acetylcholine release.

The majority of vagal afferent nerves in the lungs are nociceptors that are adept at sensing the type of tissue injury and inflammation that occurs in the lungs in COPD. In addition, stretch sensitive afferent nerves are present in the lungs and can be activated by the tissue distention that occurs during eupneic (normal) breathing. The pattern of action potential discharge in these fibers depends on the rate and depth of breathing, the lung volume at which respiration is occurring, and the compliance of the lungs. Therefore, because COPD patients exhibit impaired breathing, the activity of nociceptive and mechano-sensitive afferent nerves is grossly altered in patients with COPD. The distortion in vagal afferent nerve activity in COPD may lead to situations where these responses are out of sync with the body's needs.

There may be clinical advantage for therapeutic treatments of the present disclosure to alleviate airway smooth muscle constriction, mucus production and other pulmonary symptoms before or during exacerbation events, such as acute exacerbations of COPD and/or asthma attacks, by reversibly blocking signals from travelling along target nerves, such as vagal nerves.

### SUMMARY

The present disclosure, in its various aspects, meets an ongoing need in the medical field, such as the field of neuromodulation, for systems and methods for reversibly blocking an electrical signal from travelling along a target nerve. In particular, the present disclosure provides systems and methods for relieving a pulmonary symptom by reversibly blocking an electrical signal from travelling along the vagus nerve or internal branch of the superior laryngeal nerve

In one aspect, the present disclosure relates to a system, comprising: an energy transmitting element, and a plurality of electrodes disposed about an inner surface of the energy transmitting element, wherein the energy transmitting element is configured to be disposed about a portion of a target nerve such that at least one electrode of the plurality of electrodes contacts the target nerve; and a controller electrically coupled to each electrode of the plurality of electrodes. The energy transmitting element may be moveable between a first configuration and a second configuration. At least one electrode of the plurality of electrodes may be configured to contact the target nerve when the energy transmitting element is in the second configuration. The energy transmitting element may include a coiled lead, a cuff moveable between a first unrolled configuration and a second rolled configuration, a hook moveable from between a first extended configuration and a second retracted configuration, and/or a cassette moveable between a first open configuration and a second closed configuration. Each electrode of the plurality of electrodes may be configured to act as one or more of a sensing electrode, mapping electrode, pacing electrode, stimulating electrode and ablation electrode. The controller may include an electrical activity processing system configured to measure an intrinsic electrical activity of the target nerve, wherein the intrinsic electrical activity is delivered to the electrical activity processing system from at least one electrode of the plurality of electrodes. In addition, or alternatively, controller may include an energy source configured to deliver treatment energy to each electrode of the plurality of electrodes. In addition, or alternatively, the controller may include an energy source configured to deliver treatment energy to the electrode or electrodes of the plurality of electrodes that measured an intrinsic electrical activity of the target nerve. In addition, or alternatively, the controller may be configured to deliver treatment energy sufficient to reversibly reduce an ability of the target nerve to send an electrical signal. The controller may further include a sensor configured to detect a body parameter, and the controller may further include an energy source configured to deliver treatment energy when the body parameter is detected. The energy transmitting element may also include an antenna configured to send and receive electrical signals from each electrode of the plurality of electrodes. The antenna may be configured for external power delivery.

In another aspect, the present disclosure relates to a system, comprising: an energy transmitting element; a plurality of electrodes disposed about an outer surface of the energy transmitting element, wherein the energy transmitting element is configured to be disposed along a portion of a target nerve such that at least one electrode of the plurality of electrodes contacts the target nerve; and a controller electrically coupled to each electrode of the plurality of electrodes. The energy transmitting element may include a lead. The system my further include a cuff moveable between a first configuration and a second configuration, wherein the cuff is configured to be disposed about the energy transmitting element and the target nerve when in the second configuration.

In yet another aspect, the present disclosure relates to a method of treating a target nerve, comprising: positioning an energy transmitting element around or adjacent to a target nerve, wherein the energy transmitting element includes a plurality of electrodes disposed about a surface thereof; determining which electrode, or electrodes, of the plurality of electrodes are in contact with the target nerve; and delivering treatment energy from the electrode or electrodes that are in contact with the target nerve, wherein the treatment energy is sufficient to at least partially relieve a pulmonary symptom. The treatment energy may reduce an ability of the target nerve to send an electrical signal. The treatment energy may be delivered following the detection of a body parameter. The method may further comprise monitoring the body parameter, and altering the treatment energy based on the measured body parameter.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1 is a schematic view of an airway and a cholinergic pathway.

FIG. 2 is a schematic view of an airway and afferent nerves.

FIGS. 3A-3B illustrate an energy transmitting cuff in open (FIG. 3A) and closed (FIG. 3B) configurations, according to an embodiment of the present disclosure.

FIGS. 4A-4B illustrate an energy transmitting coiled lead that may be directly attached to a controller (FIG. 4A), or includes an embedded circuit (FIG. 4B) for wirelessly communicating with the controller, according to embodiments of the present disclosure.

FIGS. 5A-5B illustrate an energy transmitting hook which is moveable between an extended configuration (FIG. 5A) and a retracted configuration (FIG. 5B), according to an embodiment of the present disclosure.

FIGS. 6A-6B illustrate an energy transmitting cassette in open (FIG. 6A) and closed (FIG. 6B) configurations, according to an embodiment of the present disclosure.

FIG. 7 illustrates an energy transmitting lead according to an embodiment of the present disclosure.

FIG. 8 illustrates a cuff disposed around the energy transmitting lead of FIG. 7, according to an embodiment of the present disclosure.

FIGS. 9A-9B illustrate an energy transmitting paddle lead in closed (FIG. 9A) and open (FIG. 9B) configurations, according to an embodiment of the present disclosure.

FIG. 10 illustrates the use of a handheld device to signal a controller to deliver energy to electrode(s) of an energy transmitting element, according to an embodiment of the present disclosure.

FIG. 11A illustrates the energy transmitting coiled lead of FIG. 4A disposed around a bronchus and vagus nerve of the lung, according to an embodiment of the present disclosure.

FIG. 11B illustrates the energy transmitting cuff of FIG. 3B disposed around the bronchi and vagus nerves of the lung, according to an embodiment of the present disclosure.

FIG. 12 illustrates the energy transmitting coiled lead of FIG. 4A disposed around the vagus nerve, according to an embodiment of the present disclosure.

FIG. 13 illustrates a coiled lead disposed around the internal branch of the superior laryngeal nerve, according to an embodiment of the present disclosure.

FIG. 14 illustrates the coiled lead of FIG. 13 electrically connected to a controller, in accordance with an embodiment of the present disclosure.

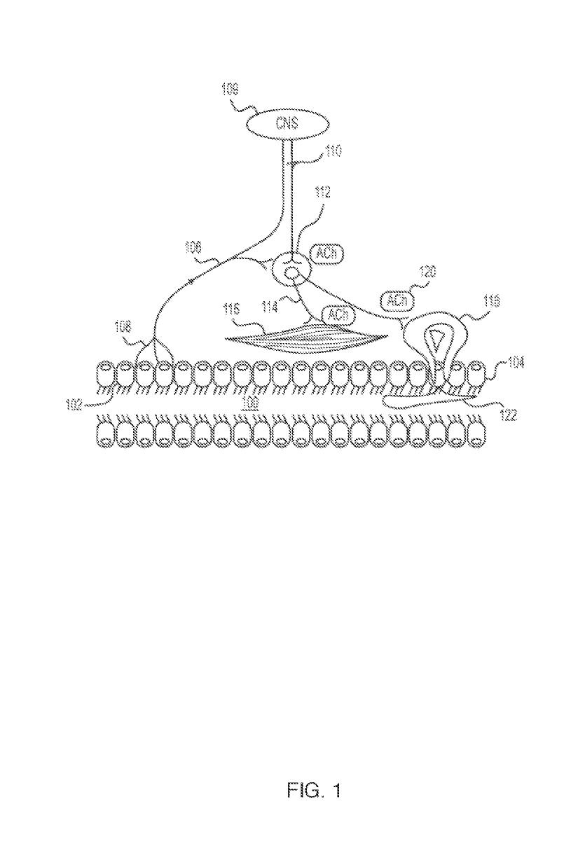

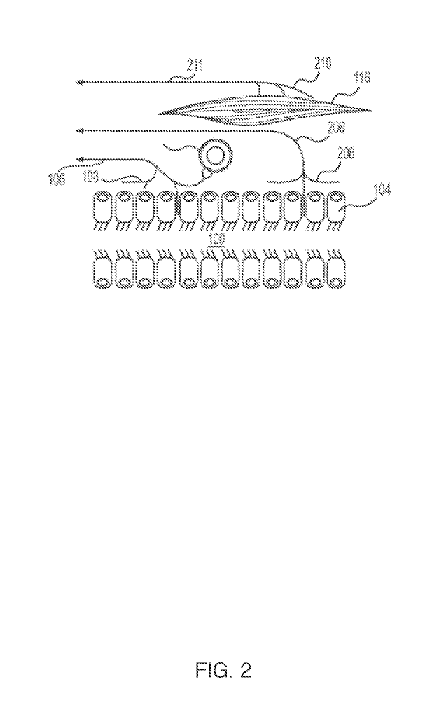

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to systems and methods for reversibly blocking an electrical signal from travelling along the vagus nerve or internal branch of the superior laryngeal nerve to relieve pulmonary symptoms, it should be appreciated that such systems and methods may be used to establish a reversible conduction block along a variety of nerves and nervous systems to treat a variety of acute or chronic symptoms. For example, a reversible conduction block of various sympathetic nerves may reduce or eliminate symptoms of pain and/or vascular tone, while blocking motor nerves may provide relief of movement disorders.

As used herein, the term “distal” refers to the end farthest away from a medical professional when introducing a device into a patient, while the term “proximal” refers to the end closest to the medical professional when introducing a device into a patient.

The systems and methods of the present disclosure are described herein with particular exemplary reference to relieving pulmonary symptoms (e.g., airway smooth muscle contraction (ASM), mucus production, etc.) by reversibly blocking parasympathetic nerves that traverse along the bronchi of the lung. It should be appreciated that reversibly blocking such nerves may reduce or control other reflexes, including, for example, chronic coughing, dyspnea and dynamic hyperinflation.

In one embodiment, the present disclosure provides an energy transmitting element comprising a plurality of electrodes spaced about an inner surface thereof. The energy transmitting element may include a variety of shapes or configurations designed to be disposed around or alongside a target nerve such that one or more of the plurality of electrodes are placed in contact with, or in the vicinity of the target nerve. To this end, the electrodes may be spaced both axially and longitudinally about the surface of the energy transmitting element. Each electrode of the plurality of electrodes may be electrically coupled to a controller by one or more conducting wires. Each of the electrodes may be configured to act as one or more of a sensing electrode, mapping electrode, pacing electrode, stimulating electrode and ablation electrode.

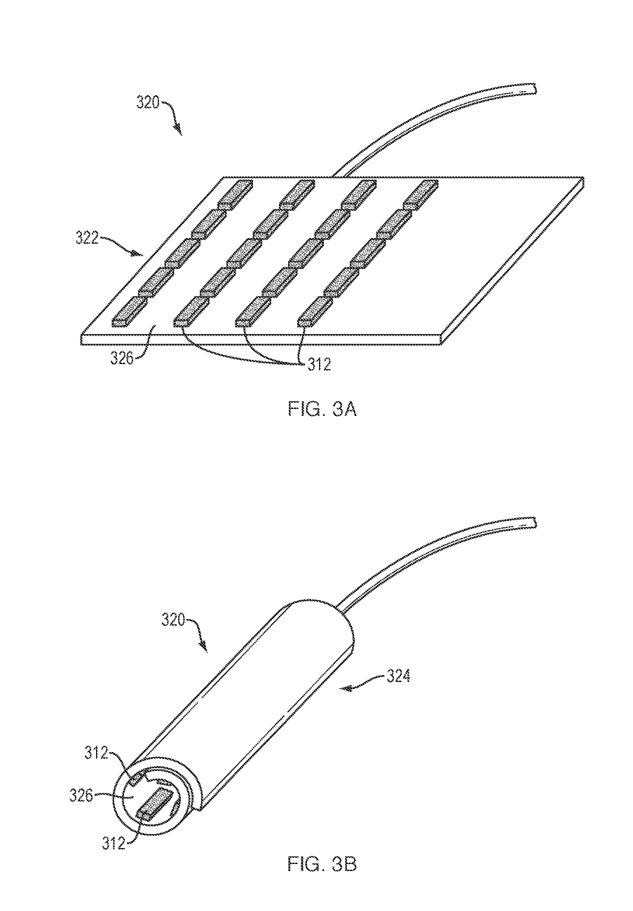

Referring to FIGS. 3A-3B, in one embodiment, the energy transmitting element may include a cuff 320 configured to move between a first (i.e., planar or unrolled) configuration 322 and a second (i.e., circular or rolled) configuration 324. A plurality of electrodes 312 may be distributed about an inner surface 326 of the cuff 320. For example, the electrodes 312 may be arranged in four rows of five electrodes when in the first configuration 322, such that each row of electrodes is arranged at 90° intervals when the cuff moves to the second configuration 324. In an embodiment in which the cuff is disposed around a target nerve (i.e., when the cuff is in the second configuration), the distribution of electrodes may allow consistent/even contact along the outer surface of the target nerve along the cuff length. Alternatively, in an embodiment in which the cuff is disposed around an anatomical feature which the target nerve runs along, such as a lung bronchus, the distribution of electrodes may allow a portion of those electrodes to be in contact with the surface of the target nerve. The cuff may further include a plurality of conducting wires (not depicted), in which a first end of the plurality of conducting wires is electrically coupled to a different one of the plurality of electrodes and a second end of the plurality of conducting wires is electrically coupled to a controller (not shown). In addition, or alternatively, the cuff 320 may be inflatable or include an inflatable member (not shown) configured to press the inner surface 326 against the target nerve (or anatomical feature) to maintain contact between the electrodes and target nerve.

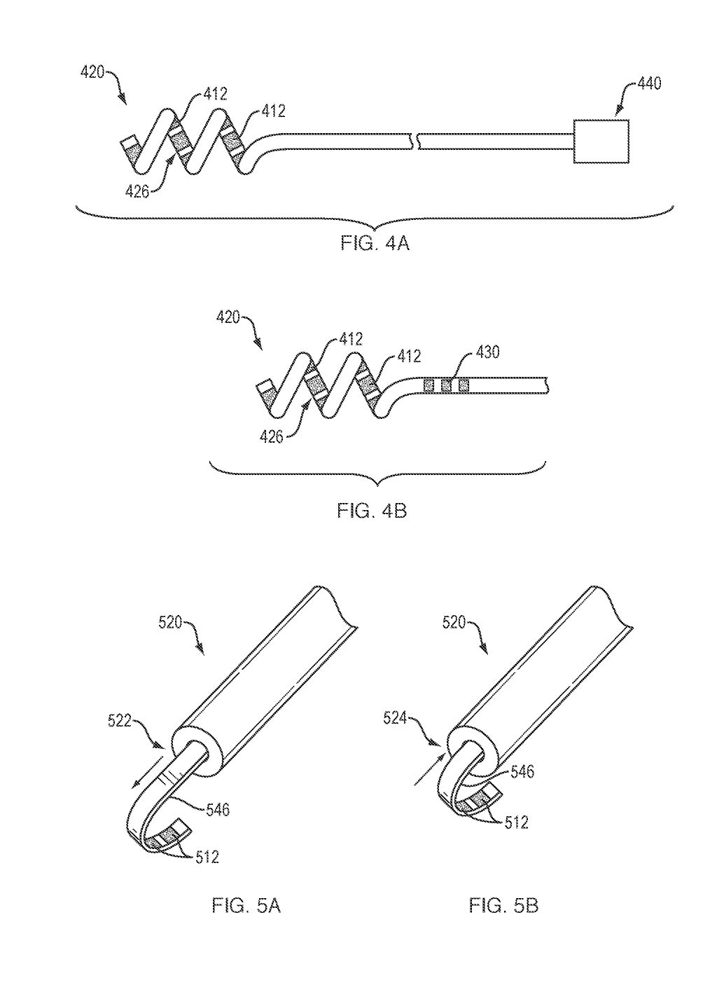

Referring to FIGS. 4A-4B, in one embodiment, the energy transmitting element may include a coiled lead 420 (e.g., coiled electrode, spiral lead, etc.) having a plurality of electrodes 412 distributed about an inner surface 426 of the winding (or windings) of the coiled lead. For example, electrodes 412 may be arranged at 90° intervals along an inner surface 426 of the windings. In an embodiment in which the coiled lead is disposed around a target nerve, the distribution of electrodes may allow consistent/even contact along the outer surface of the target nerve along the length of the lead. Alternatively, in an embodiment in which the coiled lead is disposed around an anatomical feature which the target nerve runs along, such as a lung bronchus, the distribution of electrodes may allow a portion of those electrodes to be in contact with the surface of the target nerve. The coiled lead may further include a plurality of conducting wires (not depicted), in which a first end of the plurality of conducting wires is electrically coupled to a different one of the plurality of electrodes and a second end of the conducting wire is connected to a controller 440 (FIG. 4A). Alternatively, the coiled lead 420 may include one or more embedded circuits 430 (FIG. 4B) configured to wirelessly communicate with the controller. It should also be appreciated that while the embedded circuits 430 are only depicted in FIG. 4B, any of the energy transmitting elements disclosed herein may be attached to a controller either directly or wirelessly. In addition, or alternatively, the coiled lead 420 may be inflatable or include an inflatable member (not shown) configured to press the inner surface 426 against the target nerve (or anatomical feature) to maintain contact between the electrodes and target nerve.

Referring to FIGS. 5A-5B, in one embodiment, the energy transmitting element may include a hook 520 configured to move (e.g., slide) between a first (i.e., extended) configuration 522 and a second (i.e., retracted) configuration 524. A plurality of electrodes 512 may be distributed about an inner surface 546 of the hook 520. For example, the electrodes 512 may be arranged at 30° intervals along the inner surface 546 of the hook 520. In an embodiment in which the hook is disposed around a target nerve, the distribution of electrodes may allow consistent/even contact along a portion of the outer surface of the target nerve. Once disposed around the target nerve, the hook 520 may be retracted proximally from the first 522 to second 524 configuration to more securely seat the nerve against the inner surface 546 of the hook 520. Alternatively, in an embodiment in which the hook is disposed around an anatomical feature which the target nerve runs along, such as a lung bronchus, the distribution of electrodes may allow a portion of those electrodes to be in contact with the surface of the target nerve. As above, the hook may be retracted proximally from the first to second configuration to more securely seat the anatomical feature against the inner surface of the hook. The hook may further include a plurality of conducting wires (not depicted), in which a first end of the plurality of conducting wires is electrically coupled to a different one of the plurality of electrodes and a second end of the conducting wire is connected to a controller (not shown).

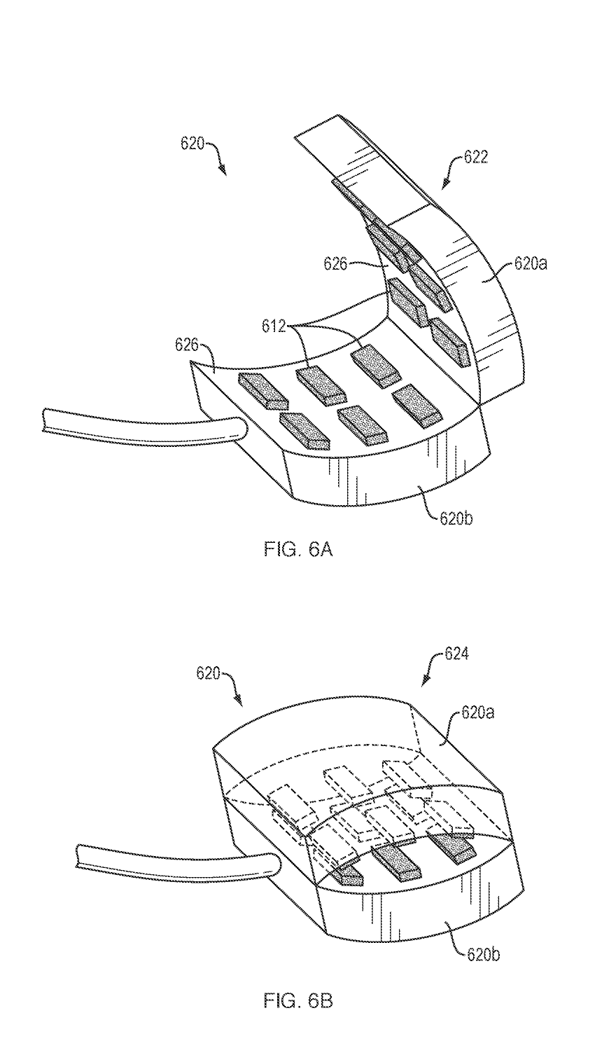

Referring to FIGS. 6A-6B, in one embodiment, the energy transmitting element may include a cassette 620 configured to move between a first (i.e., open) configuration 622 and a second (i.e., closed) configuration 624. A plurality of electrodes 612 may be distributed about an inner surface 626 of the top and bottom portions 620a, 620b of the cassette 620. For example, the top portion 620a of the cassette 620 may include two rows of electrodes 612 and the bottom portion 620b may include an additional two rows of electrodes 612, such that when the cassette 620 moves to the second configuration 624 the opposing rows of electrodes 612 provide 360° of coverage of a target nerve (or anatomical structure) disposed within the cassette. In one embodiment, the plurality of electrodes 612 on the inner surface 626 of the top portion 620a may be staggered from the plurality of electrodes on the bottom portion 620b such that direct conduction (i.e., energy delivery) between electrodes does not occur. Alternatively, the plurality of electrodes 612 may be distributed about an inner surface 626 of either the top or bottom portions 620a, 620b, but not both. In an embodiment in which the cassette is closed around a target nerve, the distribution of electrodes may allow consistent/even contact along the outer surface of the target nerve along the width of the cassette. Alternatively, in an embodiment in which the cassette is disposed around an anatomical feature which the target nerve runs along, such as a lung bronchus, the distribution of electrodes may allow a portion of those electrodes to be in contact with the surface of the target nerve. It should be appreciated that the shape or profile of the cassette may be tailored to the specific target of interest. For example, if the cassette is configured for placement around the bronchus, the inner profile of the cassette may include a circular profile corresponding to the outer diameter of the bronchus. The cassette may further include a plurality of conducting wires (not depicted), in which a first end of the plurality of conducting wires is electrically coupled to a different one of the plurality of electrodes, and a second end of the plurality of conducting wires is electrically coupled to a controller (not shown).

In another embodiment, the cassette 620 may include a securing element configured to maintain the top and bottom portions 620a, 620b of the cassette in a closed configuration around the target nerve (or anatomical feature). For example, the securing element may include a latch disposed on the top portion 620a of the cassette 620 configured to engage a corresponding post or recess disposed on the bottom portion 620b of the cassette 620. Alternatively, the top and bottom portions 620a, 620b may include corresponding apertures (e.g., suture holes) through which a suture may be tied to maintain the cassette 620 in a closed configuration.

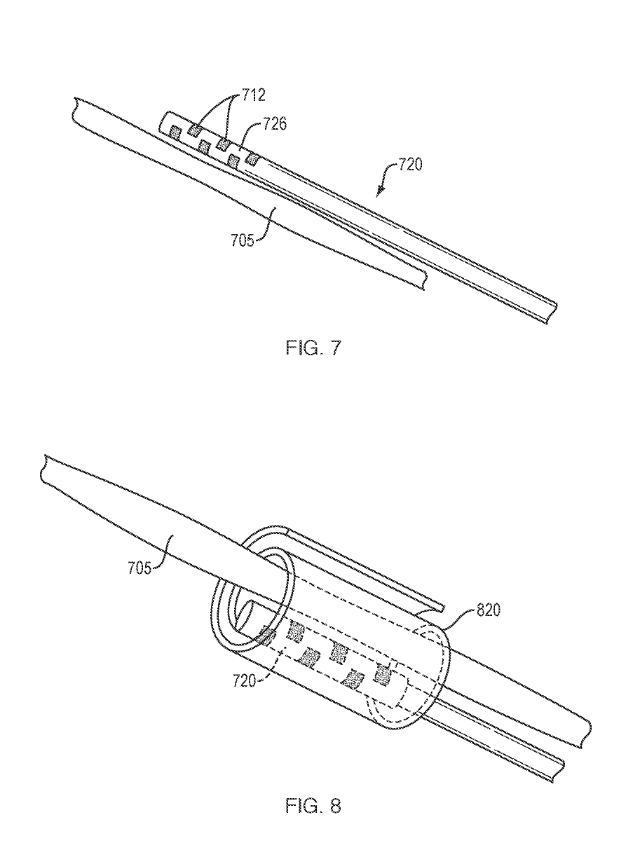

Referring to FIG. 7, in one embodiment, the energy transmitting element may include a lead 720 having a plurality of electrodes 712 distributed about an outer surface 726 thereof. The distribution of electrodes 712 ensures that at least a portion of the electrodes are placed in contact with a target nerve 705. The lead 720 may further include a plurality of conducting wires (not depicted), in which a first end of the plurality of conducting wires is electrically coupled to a different one of the plurality of electrodes, and a second end of the plurality of conducting wires is electrically coupled to a controller (not shown). As illustrated in FIG. 8, in one embodiment, the lead 720 of FIG. 7 may be maintained in position alongside the target nerve 705 with a sheath 820 configured to wrap around the lead 720 and target nerve 705. Alternatively, the lead 720 of FIG. 7 may be maintained in position alongside the target nerve 705 with a sheath 820 configured to wrap around an outer surface of lead 720 and an anatomical feature, such as a lung bronchus (not shown). In addition to maintaining the position of the lead 720 about the target nerve (or other anatomical feature), the sheath 820 may also minimize unintended non-target effects, e.g., extraneous stimulation of nearby tissues and/or organs. In addition, or alternatively, the sheath 820 may include an inflatable member (not shown) configured to press the outer surface 726 against the target nerve (or anatomical feature) to maintain contact between the electrodes and target nerve (or other anatomical feature).

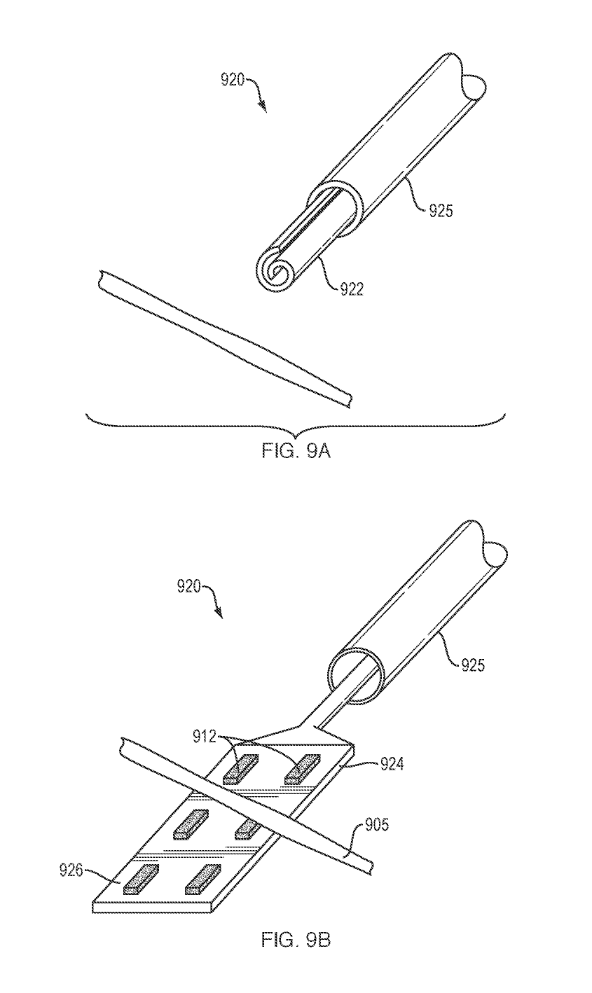

Referring to FIGS. 9A-9B, in one embodiment, the energy transmitting element may include a paddle lead (e.g., paddle electrode) 920 configured to move between a first (i.e., folded) configuration 922 and a second (i.e., unfolded) configuration 924. A plurality of electrodes 912 may be distributed about a surface 926 of the paddle lead 920. For example, the electrodes 912 may be arranged in two rows of three electrodes along a length of the paddle lead 920. The distribution of electrodes 912 ensures that at least a portion of the electrodes are placed in contact with a target nerve 905 when the paddle lead 920 is in the second configuration 924. In one embodiment, the paddle lead may wrap (e.g., fold, collapse etc.) along the long axis when in the first configuration 922 for delivery through a delivery catheter 925. Upon release from the constraint within the delivery catheter 925, the paddle lead may unfold into the second configuration and then wrap (e.g., fold, collapse etc.) along the short axis to coil around the target nerve (or anatomical feature). The paddle lead may further include a plurality of conducting wires (not depicted), in which a first end of the plurality of conducting wires is electrically coupled to a different one of the plurality of electrodes, and a second end of the plurality of conducting wires is electrically coupled to a controller (not shown). It should be appreciated that each of the embodiments illustrated in FIGS. 3-9 may include any of various numbers, arrangement, dimensions, configurations, orientations and/or angular occurrences etc. of electrodes which may be implemented and/or optimized by one of skill in the art depending on the desired outcome and application.

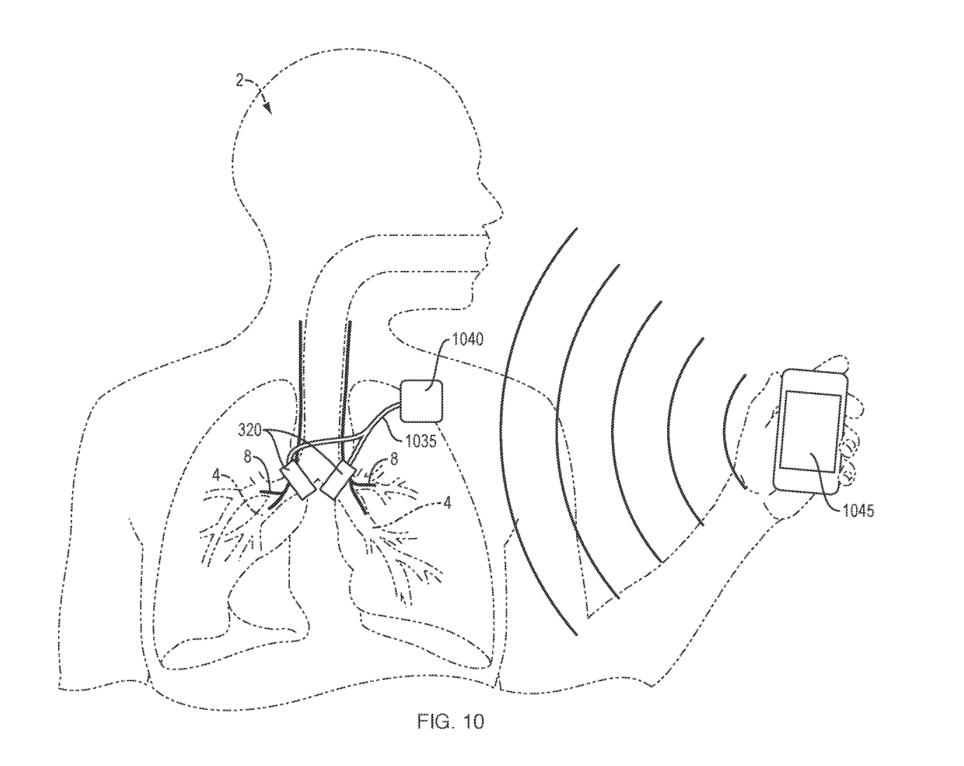

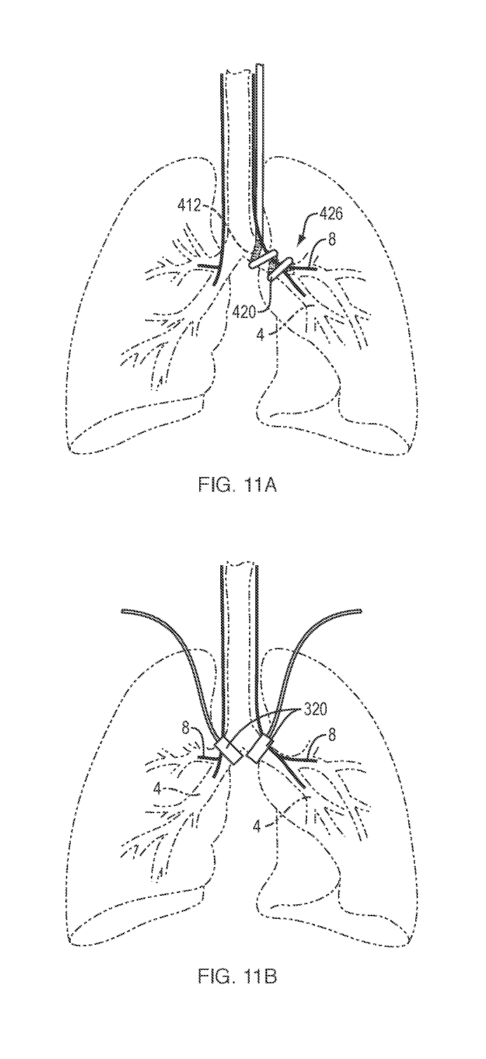

Referring to FIG. 10, in one embodiment, the electrodes of any of the energy transmitting elements disclosed herein may be electrically coupled to a controller 1040. The controller 1040 may be implanted within a subdermal pocket within the patient 2. Alternatively, the controller may be worn or carried on an external body surface of the patient (e.g., skin, clothing etc.). As discussed above, the electrodes of the energy transmitting element may be directly connected to the controller 1040 by a plurality of conducting wires 1035. For example, a first end of the plurality of conducting wires 1035 may be electrically connected to the cuff 320 of FIGS. 3A-3B disposed around the bronchi 4 and pulmonary branches of the vagus nerves 8, and a second end of the plurality of conducting wires 1035 may be advanced underneath the skin to a controller 1040 implanted within the patient's chest. Alternatively, the second end of the plurality of conducting wires may terminate in an antenna (not depicted) to wirelessly send and receive signals from an implanted or externally carried controller 1040. In one embodiment, the controller may be activated as deemed necessary by the patient. For example, the patient may activate treatment energy by “triggering” the controller to deliver treatment energy using a hand held device 1045. In addition, or alternatively, the patient may place an external power generator to their neck to deliver transcutaneous energy to electrodes implanted around the target nerve.

The controller may include an electrical activity processing system configured to measure the intrinsic electrical activity of a target nerve, or individual nerve fibers. The intrinsic electrical activity is delivered to the controller from the electrode or electrodes in contact with the target nerve and along the respective conducting wire(s). In one embodiment, identifying which electrode or electrodes sense or detect intrinsic electrical activity may allow the controller to identify which electrode(s) should be used to deliver treatment energy to the target nerve. The controller may further include an energy source, e.g., a radiofrequency (RF) generator, to deliver treatment energy to only those electrode(s) in contact with the target nerve (e.g., those that detected intrinsic electrical activity). It should be appreciated that the controller may be configured to provide a variety of energy delivery parameters based on the measured intrinsic electrical activity and/or the symptom which the treatment energy is meant to alleviate. In addition, the controller may continually or intermittently monitor the intrinsic electrical activity during (or after) the delivery of treatment energy, and vary the delivery parameter accordingly.

In another embodiment, a specific mapping protocol may be implemented at the time of implantation within the patient, or following a pre-determined time post-implantation, to identify the optimal electrode pairs for delivering treatment energy. For example, the IPG may deliver low frequency pulses of energy (e.g., less than approximately 20 Hz) to elicit action potentials and a resultant indicator of a symptom (e.g., bronchoconstriction). Higher frequency treatment energy (e.g., approximately 100 Hz to approximately 1 kHz) may then be delivered from the identified electrodes to facilitate neurotransmitter depletion blocking of the target nerve.

In another embodiment, a pulmonary symptom may be measured (i.e., monitored) during the systematic delivery of treatment energy to map (i.e., identify) the optimal electrode pairs required to achieve a reversible nerve block.

The controller may further include one or more physiological sensors configured to detect a body parameter (e.g., coughing, sneezing, wheezing and/or mucus production) indicative of a target symptom, and provide closed-loop “smart therapy” to deliver treatment energy to the electrode or electrodes previously identified as being in contact with the target nerve when an attack is detected. For example, the sensor may include an impedance sensor configured to detect or measure mucus production, airway smooth muscle (ASM) contraction, inflammation and/or elevated respiratory rate. In addition, or alternatively, the sensor may include an electrocardiogram (ECG), perfusion or blood pressure sensor configured to detect an elevated or variable heart rate, blood pressure or respiratory rate. In addition, or alternatively, the sensor could be configured to detect a change in autonomic tone, such as by detecting changes in heart rate variability (HRV). Examples of HRV parameters include standard deviation of normal-to-normal intervals (SDNN), standard deviation of averages of normal-to-normal intervals (SDANN), ratio of low-frequency (LF) to high-frequency (HF) HRV (LF/HF ratio), HRV footprint, root-mean-square successive differences (RMSSD), and percentage of differences between normal-to-normal intervals that are greater than 50 milliseconds (pNN50). In addition, or alternatively, the sensor may include an acoustic sensor configured to detect wheezing, coughing and other body sounds associated with airway obstruction or constriction. In addition, or alternatively, the sensor may include a pressure sensor configured to detect sudden pressure increases due to, e.g., coughing, wheezing or heavy breathing. For example, two or more pressure sensors may be positioned in sequence to provide an airflow sensor for measuring resistance indicative of airway constriction.

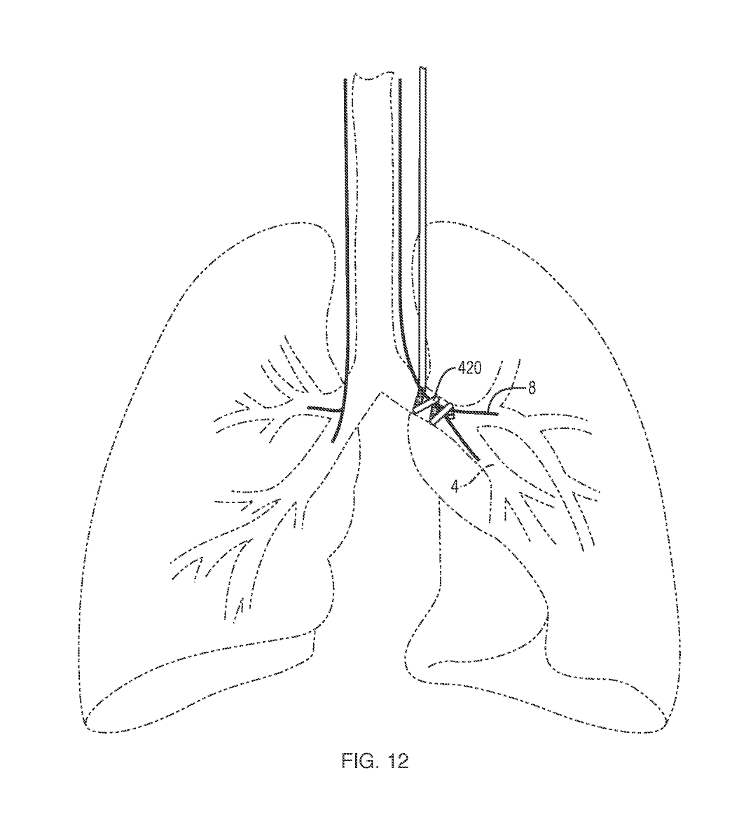

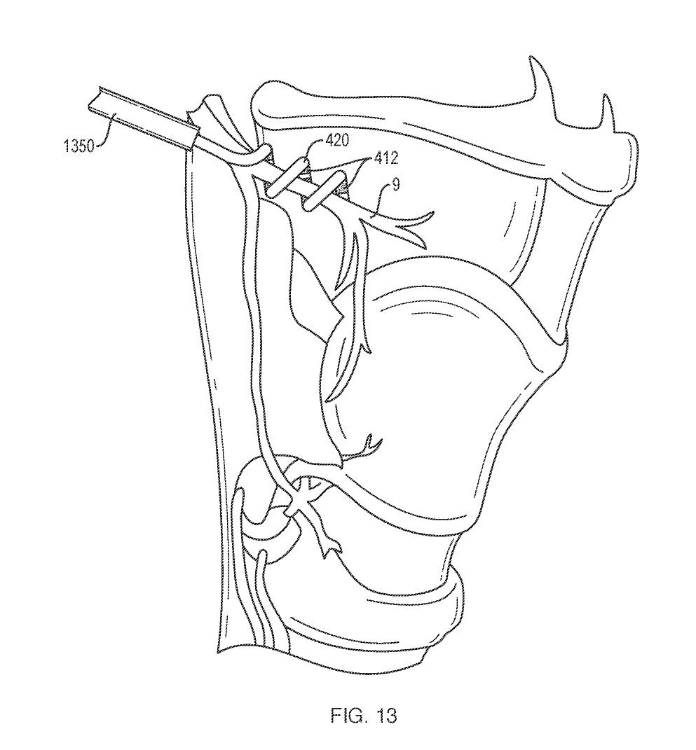

Referring to FIG. 11A, in one embodiment, an energy transmitting element such as the coiled lead 420 of FIG. 4A may be disposed around an outer surface of a first or second generation branch of a lung bronchus 4. The pulmonary branch of the vagus nerve 8 runs along an outer surface of the bronchus 4 such that a portion of the electrodes 412 on the inner surface 426 of the coiled lead are placed in contact with the bronchus 4, while a portion of the electrodes 412 are placed in contact with (or in the vicinity of) the vagus nerve 8. Referring to FIG. 11B, in another embodiment, the cuff 320 of FIGS. 3A-3B may be disposed around both bronchi 4 of the lung and the pulmonary branch of the vagus nerve 8 that runs along an outer portion of the bronchi. Similar to FIG. 11A, a portion of the electrodes (not depicted) disposed on the inner surface of the cuff 320 are placed in contact with the bronchi 4, while a portion of the electrodes 312 are placed in contact with (or in the vicinity of) the vagus nerve 8. Referring to FIG. 12, in another embodiment, the coiled lead 420 of FIG. 4A may be disposed around only the pulmonary branch of the vagus nerve 8, rather than the bronchus 4 and vagus nerve 8. It should be appreciated that any of the electrode configurations disclosed herein may be placed around one or both bronchi and/or one or both of the vagus nerves.

It should be appreciated that any of the energy transmitting elements disclosed herein may be endoscopically or laparoscopically implanted using standard surgical methods practiced by cardiothoracic surgeons to access the thoracic cavity without the need for invasive thoracotomies. Alternatively, the energy transmitting element may be implanted by an interventional pulmonologist using a bronchoscope to access the airway, such that the energy transmitting element may be inserted through the airway and in close vicinity to the target nerve branch. It should be appreciated that the energy transmitting elements disclosed herein may be delivered using a variety of delivery tools as are known in the art, including, e.g., a bronchoscope, endoscope, laparoscope, catheter, guidewire or steerable catheter or guidewire.

In one embodiment, the treatment parameter required to establish a reversible conduction block of the vagus nerve, or specific nerve fibers of the vagus nerve, may include the delivery of kHz frequency energy. Such energy may be applied in a variety of continued or pulsed waveforms, including e.g., sinusoidal, rectangular and triangular. By comparison, establishing a neuromuscular conduction block typically requires repetitive stimulation in the range of approximately 100 to 900 Hz. For example, a treatment parameter of approximately 1 kHz to 50 kHz and approximately 1 mA to 40 mA applied to one or both branches of the vagus nerve for approximately 30 minutes may provide a near-immediate nerve block which lasts for approximately 90 minutes.

In one embodiment, the present disclosure also provides systems and methods to establish a reversible electrical nerve block to one or both internal branches of the superior laryngeal nerve (ib-SLN) as a treatment for symptoms of asthma, COPD and other pulmonary conditions. It should be appreciated that the ib-SLN protects the respiratory tract by mobilizing the glottis closure reflex during swallowing, coughing and vomiting. For this reason, conventional surgical procedures only target a unilateral transection of the ib-SLN. Bilateral damage of the ib-SLN might lead to phonation disorders and disorders of respiratory control. The reversible treatments of the present disclosure may therefore allow temporary bilateral therapy with superior therapeutic results.

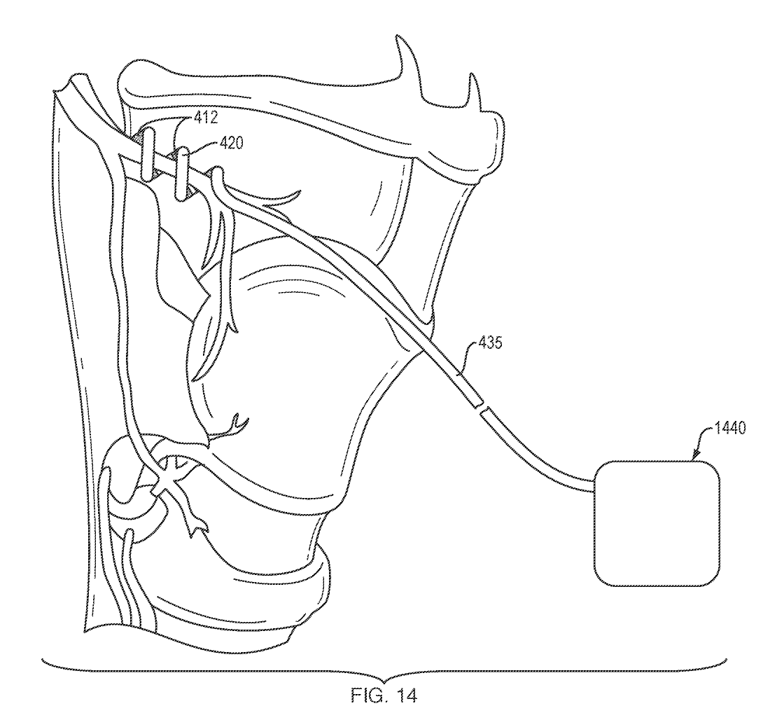

In one embodiment, the present disclosure may involve surgically implanting any of the electrode configurations disclosed herein adjacent to, or around, one or both branches of the ib-SLN, e.g., via a minimally invasive direct-visualization technique. For example, as illustrated in FIG. 13, the coiled lead 420 of FIG. 4A may be advanced to the ib-SLN through the working channel of a catheter 1350 introduced through a small incision in the patient's neck. The electrodes 412 of the coiled lead 420 may be directly or wirelessly connected to a controller carried within or on the patient's body, as discussed above. Referring to FIG. 14, in one embodiment, the electrodes 412 of the coiled lead 420 further include a plurality of conducting wires 435, in which a first end of the plurality of conducting wires 435 is electrically coupled to a different one of the electrodes 412, and a second end of the plurality of conducting wires 435 is advanced underneath the skin to a controller 1440 implanted within the patient's chest.

Energy may be delivered from the controller 1440 to the coiled lead 420 to establish a reversible nerve block. For example, a treatment parameter of approximately 1 kHz to 50 kHz and approximately 1 mA to 40 mA applied to one or both of the ib-SLN for approximately 30 minutes may provide a near-immediate nerve block which lasts for approximately 90 minutes. Alternatively, a reversible but substantially longer lasting (e.g., 6-9 months) effect may be achieved by delivering pulsed radiofrequency alternating current, e.g., approximately 480 kHz, to one branch of the ib-SLN. To avoid the potential phonation and respiratory control disorder discussed above, this longer lasting treatment is not delivered to both branches of the ib-SLN. This method may further entail one or more sensors configured to provide closed-loop temperature control to ensure that the temperature of the nerve and surrounding tissue does not exceed a temperature at which irreversible damage occurs to the nerve, for example, a temperature that does not exceed 45° C.

It should be appreciated that the electrodes of any of the energy transmitting elements disclosed herein may be unipolar, bipolar or multipolar. In one embodiment, a multipolar electrode may allow “electronic repositioning” and greater selectivity over which nerve, or nerve fibers, to stimulate. Such electrodes (leads) may be formed from materials commonly used in implantable cardiac or neurostimulation electrodes (leads) and catheters, including suitable insulative materials such as e.g., ETFE, PTFE, silicone, and PU and conductive materials such as, e.g., MP35N, stainless steel, Pt—Ir, Nitinol, Elgiloy and the like.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A system, comprising:
an energy transmitting element;
a plurality of electrodes disposed about an inner surface of the energy transmitting element, wherein the energy transmitting element is configured to be disposed about a portion of a target nerve such that at least one electrode of the plurality of electrodes contacts the target nerve; and
a controller electrically coupled to each electrode of the plurality of electrodes.

2. The system of claim 1, wherein the energy transmitting element is moveable between a first configuration and a second configuration.

3. The system of claim 2, wherein at least one electrode of the plurality of electrodes is configured to contact the target nerve when the energy transmitting element is in the second configuration.

4. The system of claim 1, wherein the energy transmitting element includes a coiled lead.

5. The system of claim 1, wherein the energy transmitting element includes a cuff moveable between a first unrolled configuration and a second rolled configuration.

6. The system of claim 1, wherein the energy transmitting element includes a hook moveable from between a first extended configuration and a second retracted configuration.

7. The system of claim 1, wherein the energy transmitting element includes a cassette moveable between a first open configuration and a second closed configuration.

8. The system of claim 1, wherein each electrode of the plurality of electrodes is configured to act as one or more of a sensing electrode, mapping electrode, pacing electrode, stimulating electrode and ablation electrode.

9. The system of claim 1, wherein the controller includes an electrical activity processing system configured to measure an intrinsic electrical activity of the target nerve, wherein the intrinsic electrical activity is delivered to the electrical activity processing system from at least one electrode of the plurality of electrodes.

10. The system of claim 1, wherein the controller includes an energy source configured to deliver treatment energy to each electrode of the plurality of electrodes.

11. The system of claim 9, wherein the controller includes an energy source configured to deliver treatment energy to the electrode or electrodes of the plurality of electrodes that measured an intrinsic electrical activity of the target nerve.

12. The system of claim 10, wherein the treatment energy reduces an ability of the target nerve to send an electrical signal.

13. The system of claim 11, wherein the controller further includes a sensor configured to detect a body parameter, and wherein the controller includes an energy source configured to deliver treatment energy to the electrode or electrodes of the plurality of electrodes that measured an intrinsic electrical activity of the target nerve when the body parameter is detected.

14. A system, comprising:
an energy transmitting element;
a plurality of electrodes disposed about an outer surface of the energy transmitting element, wherein the energy transmitting element is configured to be disposed along a portion of a target nerve such that at least one electrode of the plurality of electrodes contacts the target nerve; and
a controller electrically coupled to each electrode of the plurality of electrodes.

15. The system of claim 14, wherein the energy transmitting element includes a lead.

16. The system of claim 14, further comprising a cuff moveable between a first configuration and a second configuration, wherein the cuff is configured to be disposed about the energy transmitting element and the target nerve when in the second configuration.

17. A method of treating a nerve, comprising:
positioning an energy transmitting element around or adjacent to a target nerve, wherein the energy transmitting element includes a plurality of electrodes disposed about a surface thereof;
determining which electrode, or electrodes, of the plurality of electrodes are in contact with the target nerve; and
delivering treatment energy from the electrode or electrodes that are in contact with the target nerve, wherein the treatment energy is sufficient to at least partially relieve a pulmonary symptom.

18. The method of claim 17, wherein the treatment energy reduces an ability of the target nerve to send an electrical signal.

19. The method of claim 17, wherein the treatment energy is delivered following the detection of a body parameter.

20. The method of claim 19, further comprising monitoring the body parameter, and altering the treatment energy based on the measured body parameter.
